(19) United States
(12) Patent Application Publication
(10) Pub. No.: US 2018/0250154 A1
(43) Pub. Date: Sep. 6, 2018

(54) BLISTER PROTECTOR STICKER

(71) Applicants: Gregg Domanico, Hillsborough, CA (US); Sharon Domanico, Hillsborough, CA (US); Jack Domanico, Hillsborough, CA (US)

(72) Inventors: Gregg Domanico, Hillsborough, CA (US); Sharon Domanico, Hillsborough, CA (US); Jack Domanico, Hillsborough, CA (US)

(21) Appl. No.: 15/841,630

(22) Filed: Dec. 14, 2017

Related U.S. Application Data
(63) Continuation-in-part of application No. PCT/US2017/046855, filed on Aug. 15, 2017.
(60) Provisional application No. 62/381,286, filed on Aug. 30, 2016.

(51) Int. Cl. A61F 5/30; A61F 13/06; A61F 13/00

(52) U.S. Cl. CPC A61F 5/30; A61F 13/066; A61F 13/00059; A61F 13/068; A61F 13/067

(57) ABSTRACT

A blister protector sticker that adheres to and protects an individual's foot, including a base layer of closed cell polyvinyl chloride foam, an adhesive layer of hypoallergenic acrylate adhesive operatively attached thereto, and a removable layer removably attached to the adhesive layer, the blister protector sticker having a shape that protects and shields a portion of skin of a foot from blisters. A method of protecting an individual's foot from blisters and irritation, by removing a removable layer from the blister protector sticker, attaching the adhesive layer of the blister protector sticker to an area of skin of an individual's foot at risk of blister formation or irritation, and protecting the area of skin covered by the blister protector sticker from forming a blister or irritation.

### BACKGROUND OF THE INVENTION

### 1. Technical Field

The present invention relates to adhesive blister protectors and coverings. More specifically, the present invention relates to disposable blister protectors for feet.

### 2. Background Art

Blisters are formed on the skin when fluid (such as serum) collects in pockets under the top layer of skin, and commonly occur due to rubbing of shoes against the foot. They can be very painful and make it hard for an individual to walk in their shoes. Therefore, many people seek to protect their feet from blisters forming as well as provide cushioning and protection once a blister has occurred. Such protection can come from socks or other coverings for the foot, tape, or bandages.

U.S. Pat. No. 4,296,499 to Patterson, et al. discloses a foot covering article adapted to be worn over the bare foot and directly underneath a sock for the purpose of preventing the formation of blisters when engaging in athletic activities and the like. The article is made of a single, porous, non-woven film-fibril sheet of plexifilamentary strands, wherein the sheet is consolidated by a large number of very small point bonds, and includes a plurality of minute perforations extending through the thickness of the sheet. The sheet is formed into a foot enveloping shape comprising a bottom portion which covers the sole of the wearer's foot, side portions extending upwardly from the bottom portion, a heel portion which covers the heel of the wearer's foot, and a front portion which covers at least the forward portion of the top of the wearer's foot. The article has a smooth inner surface so that there is minimal friction between the wearer's foot and the article, and an outer surface which is rough, relative to the character of the inner surface, so that stability between the article and either the sock or shoe which is adjacent thereto is enhanced. An important property of the sheet material is that the degree of friction which is present between its inner surface and the wearer's foot decreases as the material is repeatedly crumpled. Thus, the blister preventative character of the article increases with use.

U.S. Pat. No. 4,671,266 to Lengyel, et al. discloses a blister bandage including at least one ply of an aerated latex micronized hydroentangled fabric, having an adhesive disposed on at least one surface of the fabric. The bandage has sufficient hydrophobicity in the fabric to be a barrier to liquid borne bacteria while preserving comfort, air permeability, and flexibility therein. In addition to the above properties, the bandage is sterilizable and keeps the skin beneath the bandage dry.

U.S. Pat. No. 6,916,967 to Wright, et al. discloses an adhesive bandage for protecting blisters or wounds, or for preventing chaffing or the formation of blisters. In one embodiment, the bandage includes a protective layer for covering the blister or area of skin to be protected, and an adhesive layer surrounding the protective layer. The opposite side of the bandage contains a low-friction layer or surface and a separate sheet of low-friction material adjacent to but movable with respect to the low-friction layer or surface. The sheet of low-friction material is attached to the bandage in such a way that it can move with respect to the bandage in response to frictional forces, thereby minimizing a transfer of frictional forces from clothing or the like to the bandage. A method of treating blisters, as well as a method of preventing chaffing and the formation of blisters are also disclosed.

The products currently available to prevent blisters cannot stay on the skin for long periods of time, cannot remain flexible, and can leave residue on the skin when removed. Therefore, there remains the need for a blister protector that is durable, easy to use, and effectively protects the foot of the individual wearing the blister protector.

### SUMMARY OF THE INVENTION

The present invention provides for a blister protector sticker that adheres to and protects an individual's foot, consisting of a base layer of closed cell polyvinyl chloride foam, an adhesive layer of hypoallergenic acrylate adhesive operatively attached thereto, and a removable layer removably attached to the adhesive layer, the blister protector sticker having a shape that protects and shields a portion of skin of a foot from blisters.

The present invention also provides for a method of protecting an individual's foot from blisters and irritation, by removing a removable layer from a blister protector sticker consisting of a base layer of closed cell polyvinyl chloride foam, an adhesive layer of hypoallergenic acrylate adhesive operatively attached to the base layer, and a removable layer removably attached to the adhesive layer, the blister protector sticker having a shape that protects and shields a portion of skin of a foot from blisters, attaching the adhesive layer of the blister protector sticker to an area of skin of an individual's foot at risk of blister formation or irritation, and protecting the area of skin covered by the blister protector sticker from forming a blister or irritation.

### DESCRIPTION OF THE DRAWINGS

Other advantages of the present invention are readily appreciated as the same becomes better understood by reference to the following detailed description when considered in connection with the accompanying drawings wherein:

FIG. 1 is a cross-sectional view of the blister protector sticker of the present invention;

FIG. 2A is a top view of the blister protector sticker in the shape of a rectangle with representative dimensions, FIG. 2B is a top view of the blister protector sticker in the shape of a heart with representative dimensions, FIG. 2C is a top view of the blister protector sticker in the shape of a butterfly with representative dimensions, FIG. 2D is a top view of the blister protector sticker in the shape of a star, FIG. 2E is a top view of the blister protector sticker in the shape of a pumpkin, FIG. 2F is a top view of the blister protector sticker in the shape of a circle, FIG. 2G is a top view of the blister protector sticker in the shape of a square, FIG. 2H is a top view of the blister protector sticker in the shape of a triangle, and FIG. 2I is a top view of the blister protector sticker in the shape of a flower;

FIG. 3 is a top view of blister protector stickers on a sheet of the removable layer;

FIG. 4 is a side view of the blister protector sticker applied to a foot; and

FIG. 5 is a top view of representative dimensions of blister protector stickers on a sheet of the removable layer.

### DETAILED DESCRIPTION OF THE INVENTION

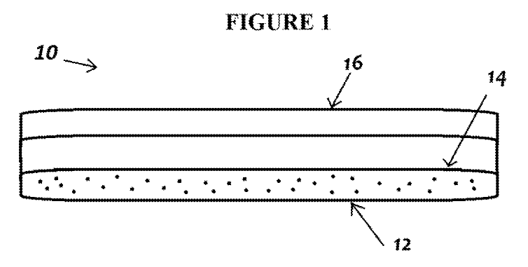

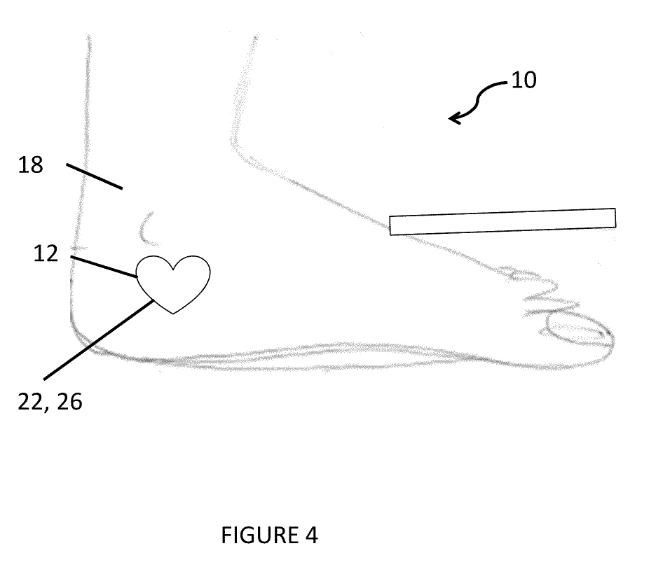

The present invention generally provides for a blister protector sticker, generally indicated at 10 in the FIGURES, that adheres to and protects and shields a portion of skin of an individual's foot 18. The blister protector sticker 10 includes a base layer 12, an adhesive layer 14 operatively attached thereto, and a removable layer 16 removably attached to the adhesive layer 14, the blister protector sticker 10 being in a shape 22 that covers a portion of the foot 18, as shown in FIG. 1 and FIG. 4. The blister protector sticker 10 is able to stay on the skin once applied for long periods of time, remains flexible while wearing, and can easily be removed without leaving any residue on the skin.

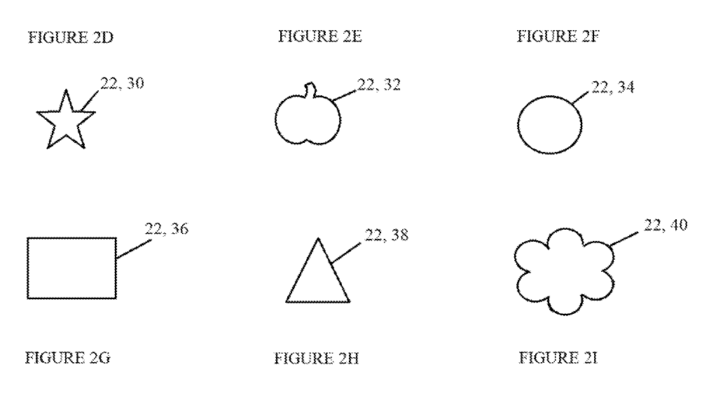

The blister protector sticker 10 can be made in many different decorative shapes 22, such as in the shape of a heart (26 in FIG. 2B), butterfly (28 in FIG. 2C), star (30 in FIG. 2D), pumpkin (32 in FIG. 2E), square (36 in FIG. 2G), rectangle (24 in FIG. 2A), triangle (38 in FIG. 2H), flower (40 in FIG. 2I), or circle (34 in FIG. 2F). The blister protector sticker 10 can be made in all different sizes to cover different areas of an individual's foot, and can be made and sized to specifically cover the toes, heel, arch, ankle area, portion of the sole, or other specific areas prone to blisters. An example of representative dimensions are shown in FIGS. 2A-2C. The blister protector sticker 10 can be made in any color, have logos for companies or sports teams, or include decorative designs. The blister protector sticker 10 can be applied directly to an individual's foot as well as socks, tights, or any other foot covering in order to provide blister protection. The blister protector sticker 10 does not include additional elements besides those recited herein.

The base layer 12 is preferably a foam material that provides cushioning to the foot and is flexible to allow the foot to bend and walk with ease. Preferably, the base layer 12 is thin enough such that the blister protector sticker 10 can easily fit inside an individual's shoes and be worn comfortably. The base layer 12 can be made from a foam sheet, tape, rubber, plastic, cloth fabric, or paper and cut to a desired size. Most preferably, the foam is 3M™ Microfoam elastic surgical tape, product number 9777L. 9777L has been shown to have no primary skin irritation, no delayed dermal contact sensitization, and no allergic contact dermatitis in testing in both humans and animals, and is thus considered hypoallergenic. The 9777L tape includes closed cell polyvinyl chloride foam as the base layer 12 (preferably 0.8 mm thick), and a hypoallergenic acrylate adhesive as the adhesive layer 14. The foam material can be made from any suitable material, such as, but not limited to, polyolefin, polyethylene, polyurethane, rubber foams, vinyl foams and combinations thereof. The foam can be closed cell or open cell. The foam used can allow the foot to breathe while wearing the blister protector sticker 10. The base layer 12 is a unitary and flat structure, i.e. there are no segments or breaks in the foam, no ridges, or additional elements between the base layer 12 and the adhesive layer 14.

The adhesive layer 14 is operatively attached to the base layer 12 in any suitable manner. The adhesive layer 14 is strong enough to maintain the blister protector sticker 10 on the foot during use but can also easily be removed by the individual when they want to remove the blister protector sticker 10 and does not leave a residue on the skin of the foot. The adhesive layer 14 can cover all or a portion of the base layer 12. The adhesive layer 14 can be a pressure sensitive adhesive that forms a bond due to the application of light pressure of a foot on the adhesive layer 14. The adhesive layer 14 can be made of an epoxy adhesive, cyanoacrylates, water-based adhesives, double-sided bonding tapes, 3M™ gripping material, masking/duct tapes, and adhesive transfer tapes, or any other suitable adhesive. The adhesive layer 14 is designed to retain contact with the foot 18 in dry and moist conditions.

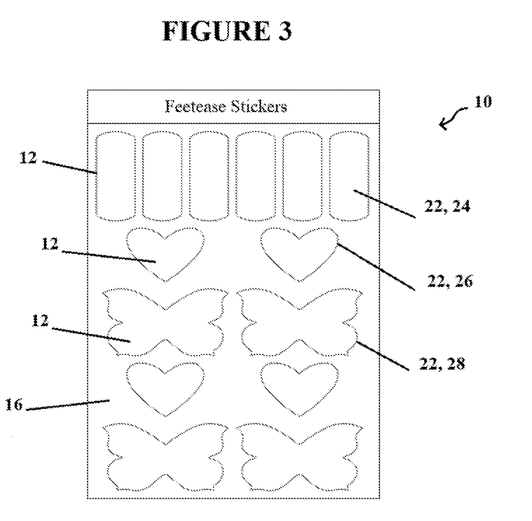

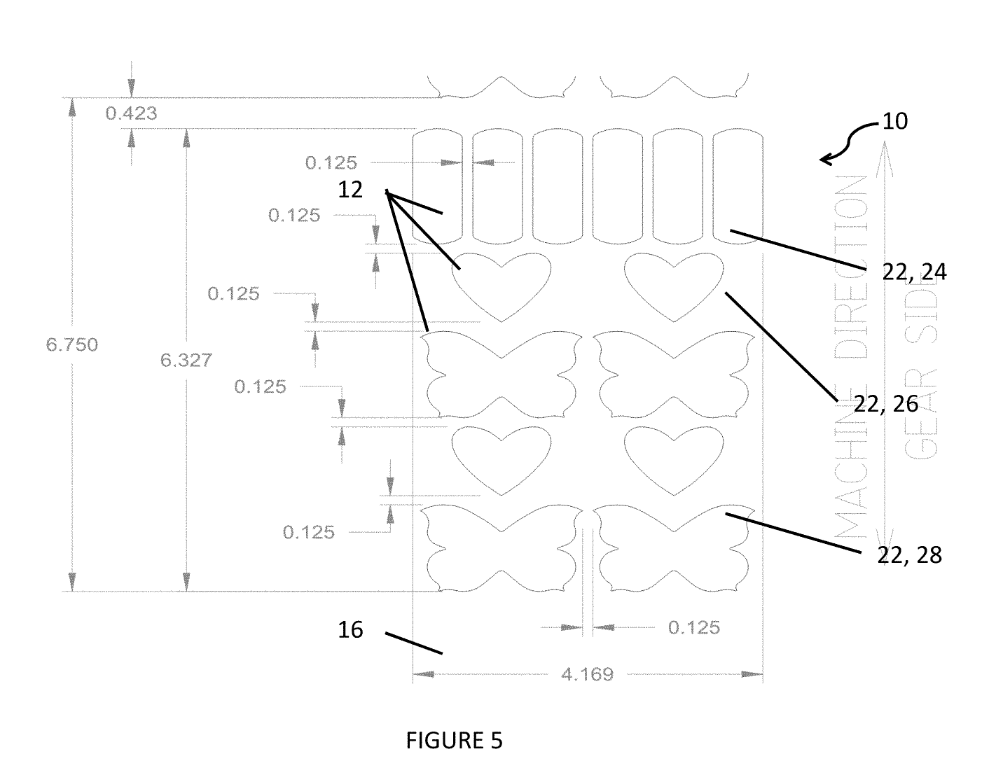

The removable layer 16 can easily be removed and peeled off from the adhesive layer 14 by an individual when they want to apply the blister protector sticker 10 to their feet. The removable layer 16 protects the adhesive layer 14 until the blister protector sticker 10 is ready to be used. The removable layer 16 is generally in the shape of the base layer 12, but can be in any other shape and can cover areas of the base layer 12 that have the adhesive layer 14. The size of the removable layer 16 can be the same as the base layer 12 or slightly larger to aid in removing. The removable layer 16 can also be a sheet 20 with multiple blister protector stickers 10 applied thereon, as shown in FIG. 3 and in FIG. 5 with representative dimensions (and includes multiple different shapes 22 of the blister protector stickers 10). The removable layer 16 can be made out of paper, plastic, plastic coated paper, wax paper, or any other suitable material that allows the removable layer 16 to easily be released from the adhesive layer 14.

The present invention also provides for a method of protecting an individual's foot from blisters and irritation, by removing a removable layer 16 from the blister protector sticker 10 described above having a base layer 12, an adhesive layer 14 operatively attached thereto, and a removable layer 16 removably attached to the adhesive layer 14, attaching the adhesive layer 14 of the blister protector sticker 10 to an area of skin of an individual's foot, and protecting the area of skin covered by the blister protector sticker 10 from forming a blister or irritation. Most preferably, the blister protector sticker 10 includes the base layer 12 of closed cell polyvinyl chloride foam and the adhesive layer of hypoallergenic acrylate adhesive as the adhesive layer 14, and most preferably is made of the microfoam elastic surgical tape 9777L (3M™). Alternatively, any other materials described above can be used. The blister protector sticker 10 securely attaches to the foot, as shown in FIG. 4. The individual can subsequently put their shoes on, i.e. the shoes can be worn over the blister protector sticker 10. When they do not want to use the blister protector sticker 10 anymore, they can easily remove it by peeling the blister protector sticker 10 off of their foot. The blister protector sticker 10 does not leave any adhesive residue when removed from the foot. Alternatively, the adhesive layer 14 can be applied to the individual's sock. The blister protector sticker 10 can also be used to protect and area of skin that already has a blister or irritation and can help relieve discomfort of the foot by providing a barrier between the foot 18 and a shoe.

The blister protector sticker 10 can be used in many different situations, such as, but not limited to, wearing athletic shoes while playing sports, breaking in new shoes, protecting sensitive skin from rubbing shoes, walking, running, dancing, hiking, and walking long distances in the military.

Throughout this application, various publications, including United States patents, are referenced by author and year and patents by number. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology, which has been used is intended to be in the nature of words of description rather than of limitation.

Obviously, many modifications and variations of the present invention are possible in light of the above teachings. It is, therefore, to be understood that within the scope of the appended claims, the invention can be practiced otherwise than as specifically described.

What is claimed is:

1. A blister protector sticker that adheres to and protects an individual's foot, consisting of:
a base layer of closed cell polyvinyl chloride foam;
an adhesive layer of hypoallergenic acrylate adhesive operatively attached to said base layer; and
a removable layer removably attached to said adhesive layer, said blister protector sticker having a shape that protects and shields a portion of skin of a foot from blisters.

2. The blister protector sticker of claim 1, wherein said shape is chosen from the group consisting of a heart, a butterfly, a star, a pumpkin, a square, a rectangle, a triangle, a flower, and a circle.

3. The blister protector sticker of claim 1, wherein said blister protector sticker is sized to fit a part of the foot chosen from the group consisting of toes, heel, arch, ankle area, and portion of sole.

4. The blister protector sticker of claim 1, wherein said base layer and said adhesive layer are made from microfoam elastic surgical tape 9777L (3M™).

5. The blister protector sticker of claim 1, wherein said adhesive layer is a pressure sensitive adhesive.

6. The blister protector sticker of claim 1, wherein said adhesive layer is removable from the foot without leaving residue on the skin.

7. The blister protector sticker of claim 1, wherein said removable layer is made of a material chosen from the group consisting of paper, plastic, plastic coated paper, and wax paper.

8. The blister protector sticker of claim 1, wherein said removable layer is in the shape and size of said base layer.

9. The blister protector sticker of claim 1, wherein said removable layer is a sheet and includes multiple blister protector stickers attached thereon.

10. A method of protecting an individual's foot from blisters and irritation, including the steps of:
removing a removable layer of a blister protector sticker consisting of a base layer of closed cell polyvinyl chloride foam, an adhesive layer of hypoallergenic acrylate adhesive operatively attached to the base layer, and a removable layer removably attached to the adhesive layer, the blister protector sticker having a shape that protects and shields a portion of skin of a foot from blisters;
attaching the adhesive layer to an area of skin of an individual's foot at risk of blister formation or irritation; and
protecting the area of skin covered by the blister protector sticker from forming a blister or irritation.

11. The method of claim 10, further including the step of removing the blister protector sticker from the individual's foot without leaving any adhesive residue.

12. The method of claim 10, wherein said method is performed during an activity chosen from the group consisting of wearing athletic shoes while playing sports, breaking in new shoes, protecting sensitive skin from rubbing shoes, walking, running, dancing, hiking, and walking long distances in the military.

13. The method of claim 10, further including the step of the individual wearing shoes over the blister protector sticker.

14. The method of claim 10, wherein said attaching step is further defined as attaching the adhesive layer to an area of skin of an individual's foot that has a blister or irritation, and further including the step of relieving discomfort of the foot.

15. The method of claim 10, wherein the shape is chosen from the group consisting of a heart, a butterfly, a star, a pumpkin, a square, a rectangle, a triangle, a flower, and a circle.
